(19) United States
(12) Patent Application Publication
(10) Pub. No.: US 2022/0202395 A1
(43) Pub. Date: Jun. 30, 2022

(54) ULTRASONIC IMAGING SYSTEM AND ULTRASONIC IMAGING METHOD

(71) Applicant: GE Precision Healthcare LLC, Wauwatosa, WI (US)

(72) Inventors: Liye Pei, Wuxi (CN); Yue Yang, Wuxi (CN); Xiaoyan Qin, Wuxi (CN); Houbing Liu, Wuxi (CN); Kejian Shi, Wuxi (CN)

(21) Appl. No.: 17/558,271

(22) Filed: Dec. 21, 2021

(30) Foreign Application Priority Data
Dec. 31, 2020 (CN) 202011630155.5

(51) Int. Cl. A61B 8/00; G06T 7/00

(52) U.S. Cl. CPC A61B 8/463; A61B 8/465; G06T 2207/30004; G06T 2207/10132; G06T 2207/30168; G06T 7/0012

(57) ABSTRACT

Methods and systems for ultrasonic imaging are provided. One method includes obtaining ultrasonic data about tissue to be imaged, generating an ultrasonic image based on the ultrasonic data, determining an anatomical region corresponding to the ultrasonic image, and generating a first visual indication reflecting the anatomical region corresponding to the ultrasonic image. The method further includes determining a quality level of the ultrasonic image, and generating a second visual indication reflecting the quality level of the ultrasonic image. The method also includes sending a first signal to a display device so that the display device simultaneously displays the ultrasonic image, the first visual indication, and the second visual indication.

### TECHNICAL FIELD

The present invention relates to the field of medical imaging, and in particular, to an ultrasonic imaging system and an ultrasonic imaging method.

### BACKGROUND

Ultrasonic imaging is a widely used imaging means. An ultrasonic imaging system can automatically identify parameters of a target object, such as the length or diameter of an anatomical structure, the volume of blood or a fluid flowing through a region over a period of time, and the speed, average speed, or peak speed of acquisition.

For ultrasound clinicians who are less skilled in operation, the quality of an acquired ultrasonic image is often unacceptable, requiring a rescan. However, lack of experience makes it impossible to determine whether the quality of the ultrasonic image is qualified. On the other hand, when it is verified that the quality of the ultrasonic image is unqualified, the rescan process is often time and labor consuming since it is untargeted.

### SUMMARY

The aforementioned deficiencies, disadvantages, and problems are solved herein, and these problems and solutions will be understood through reading and understanding of the following description.

Provided in some embodiments of the present invention is an ultrasonic imaging method, comprising: obtaining ultrasonic data about tissue to be imaged; generating an ultrasonic image based on the ultrasonic data; determining an anatomical region corresponding to the ultrasonic image, and generating a first visual indication reflecting the anatomical region corresponding to the ultrasonic image; determining a quality level of the ultrasonic image, and generating a second visual indication reflecting the quality level of the ultrasonic image; and sending a first signal to a display device, wherein the first signal is configured so that the display device simultaneously displays the ultrasonic image, the first visual indication, and the second visual indication.

Provided in some embodiments of the present invention is an ultrasonic imaging device, comprising: a probe, configured to acquire ultrasonic data; a processor, configured to obtain ultrasonic data about tissue to be imaged; generate an ultrasonic image based on the ultrasonic data; determine an anatomical region corresponding to the ultrasonic image, and generate a first visual indication reflecting the anatomical region corresponding to the ultrasonic image; determine a quality level of the ultrasonic image, and generate a second visual indication reflecting the quality level of the ultrasonic image; and send a first signal to a display device, wherein the first signal is configured so that the display device simultaneously displays the ultrasonic image, the first visual indication, and the second visual indication. The ultrasonic imaging device further comprises a display device, configured to receive a signal from the processor for display.

Provided in some embodiments of the present invention is a non-transitory computer-readable medium, storing a computer program, wherein the computer program has at least one code segment, and the at least one code segment is executable by a machine so that the machine performs the following steps: obtaining ultrasonic data about tissue to be imaged; generating an ultrasonic image based on the ultrasonic data; determining an anatomical region corresponding to the ultrasonic image, and generating a first visual indication reflecting the anatomical region corresponding to the ultrasonic image; determining a quality level of the ultrasonic image, and generating a second visual indication reflecting the quality level of the ultrasonic image; and sending a first signal to a display device, wherein the first signal is configured so that the display device simultaneously displays the ultrasonic image, the first visual indication, and the second visual indication.

It should be understood that the brief description above is provided to introduce in simplified form some concepts that will be further described in the Detailed Description of the Embodiments. The brief description above is not meant to identify key or essential features of the claimed subject matter. The scope is defined uniquely by the claims that follow the detailed description. Furthermore, the claimed subject matter is not limited to implementations that solve any disadvantages noted above or in any section of the present disclosure.

### BRIEF DESCRIPTION OF THE DRAWINGS

The present invention will be better understood by reading the following description of non-limiting embodiments with reference to the accompanying drawings, where

FIG. 1 is a schematic diagram of an ultrasonic imaging system according to some embodiments of the present invention;

FIG. 2 is a schematic diagram of an ultrasonic imaging method according to some embodiments of the present invention;

FIG. 3 is a schematic diagram of an image according to some embodiments of the present invention;

FIG. 4 is a schematic diagram of an image according to some other embodiments of the present invention;

FIG. 5 is a schematic diagram of an enlarged ultrasonic image according to some embodiments of the present invention; and

FIG. 6 is a schematic diagram of a plurality of ultrasonic images according to some embodiments of the present invention.

### DETAILED DESCRIPTION

Specific implementations of the present invention will be described in the following. It should be noted that during the specific description of the implementations, it is impossible to describe all features of the actual implementations in detail in present invention for the sake of brief description. It should be understood that in the actual implementation of any of the implementations, as in the process of any engineering project or design project, a variety of specific decisions are often made in order to achieve the developer's specific objectives and meet system-related or business-related restrictions, which will vary from one implementation to another. Moreover, it can also be understood that although the efforts made in such development process may be complex and lengthy, for those of ordinary skill in the art related to content disclosed in the present invention, some changes in design, manufacturing, production or the like based on the technical content disclosed in the present disclosure are only conventional technical means, and should not be construed as that the content of the present disclosure is insufficient.

Unless otherwise defined, the technical or scientific terms used in the claims and the description are as they are usually understood by those of ordinary skill in the art to which the present invention pertains. “First”, “second” and similar words used in the present invention and the claims do not denote any order, quantity or importance, but are merely intended to distinguish between different constituents. The term “one”, “a(n)”, or a similar term is not meant to be limiting, but rather denote the presence of at least one. The term “include”, “comprise”, or a similar term is intended to mean that an element or article that appears before “include” or “comprise” encompasses an element or article and equivalent elements that are listed after “include” or “comprise”, and does not exclude other elements or articles. The term “connect”, “connected”, or a similar term is not limited to a physical or mechanical connection, and is not limited to a direct or indirect connection.

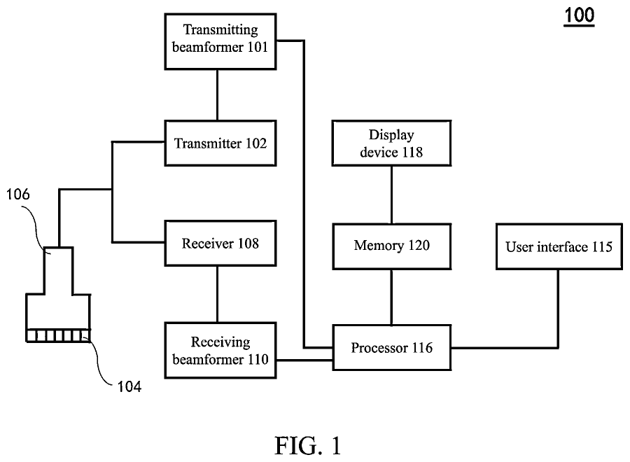

FIG. 1 is a schematic diagram of an ultrasonic imaging system 100 according to some embodiments of the present invention. The ultrasonic imaging system 100 includes a transmitting beamformer 101 and a transmitter 102, both driving elements 104 within the probe 106 to transmit ultrasonic pulse signals into the body (not shown). According to various embodiments, the probe 106 may be any type of probe including a linear probe, a curved array probe, a 1.25D array probe, a 1.5D array probe, a 1.75D array probe, or a 2D array probe. According to other embodiments, the probe 106 may also be a mechanical probe, for example, a mechanical 4D probe or a hybrid probe. The probe 106 may be configured to acquire 4D ultrasonic data, where the 4D ultrasonic data comprises information on how the volume changes over time. Each volume may include a plurality of 2D images or slices. Still referring to FIG. 1, the ultrasonic pulse signals are backscattered from structures in the body (for example, blood cells or muscle tissue) to produce echoes and return to the elements 104. The echoes are converted by the elements 104 into electrical signals or ultrasonic data, and the electrical signals are received by a receiver 108. The electrical signals representing the received echoes pass through a receiving beamformer 110 that outputs ultrasonic data. According to some embodiments, the probe 106 may include an electronic circuit to perform all or part of transmitting beamforming and/or receiving beamforming. For example, all or part of the transmitting beamformer 101, the transmitter 102, the receiver 108, and the receiving beamformer 110 may be located in the probe 106. The term “scan” or “scanning” may also be used in the present disclosure to refer to acquiring data through the process of transmitting and receiving ultrasonic signals. The terms “data” and “ultrasonic data” may be used in the present disclosure to refer to one or a plurality of datasets acquired using the ultrasonic imaging system. A user interface 115 may be configured to control operation of the ultrasonic imaging system 100. The user interface may be configured to control input of patient data, or select various modes, operations, parameters, and so on. The user interface 115 may include one or a plurality of user input devices, for example, a keyboard, hard keys, a touch pad, a touch screen, a trackball, a rotary control, a slider, soft keys, or any other user input device.

The ultrasonic imaging system 100 further includes a processor 116, which controls the transmitting beamformer 101, the transmitter 102, the receiver 108, and the receiving beamformer 110. According to various embodiments, the receiving beamformer 110 may be a conventional hardware beamformer or a software beamformer. If the receiving beamformer 110 is a software beamformer, the receiving beamformer may include one or more of the following components: a graphics processing unit (GPU), a microprocessor, a central processing unit (CPU), a digital signal processor (DSP), or any other type of processor capable of performing logical operations. The beamformer 110 may be configured to implement conventional beamforming techniques and techniques such as retrospective transmit beamformation (RTB).

The processor 116 is in electronic communication with the probe 106. The processor 116 may control the probe 106 to acquire ultrasonic data. The processor 116 controls which elements 104 are activated and the shape of a beam transmitted from the probe 106. The processor 116 is further in electronic communication with a display device 118, and the processor 116 may process the ultrasonic data into an image for display on the display device 118. For the purpose of the present disclosure, the term “electronic communication” may be defined to include wired connection and wireless connection. According to an embodiment, the processor 116 may include a central processing unit (CPU). According to other embodiments, the processor 116 may include other electronic components capable of performing processing functions, for example, a digital signal processor, a field-programmable gate array (FPGA), a graphics processing unit (GPU), or any other type of processor. According to other embodiments, the processor 116 may include a plurality of electronic components capable of performing processing functions. For example, the processor 116 may include two or more electronic components selected from a list including the following electronic components: a central processing unit (CPU), a digital signal processor (DSP), a field-programmable gate array (FPGA), and a graphics processing unit (GPU). According to another embodiment, the processor 116 may include a complex demodulator (not shown), which demodulates RF data and generates raw data. In another embodiment, the demodulation may be performed earlier in the processing chain. The processor 116 may be adapted to perform one or a plurality of processing operations on data according to a plurality of selectable ultrasound modalities. As echo signals are received, data may be processed in real time in a scanning stage. For the purpose of the present disclosure, the term “real time” is defined to include a process that is performed without any intentional delay. The real-time frame or volume rate may vary based on the site where data is acquired or the size of the volume and specific parameters used in the acquisition process. The data may be temporarily stored in a buffer (not shown) in the scanning stage, and processed in a less real-time manner in live or offline operations. Some embodiments of the present invention may include a plurality of processors (not shown) to cope with processing tasks. For example, a first processor may be configured to demodulate and decimate RF signals, while a second processor may be configured to further process data which is then displayed as an image. It should be recognized that other embodiments may use different processor arrangements. For embodiments where the receiving beamformer 110 is a software beamformer, the processing tasks belonging to the processor 116 and the software beamformer in the above text may be performed by a single processor, for example, the receiving beamformer 110 or the processor 116. Alternatively, the processing functions belonging to the processor 116 and the software beamformer may be distributed among any number of separate processing components in a different manner.

According to an embodiment, the ultrasonic imaging system 100 may continuously acquire ultrasonic data at a frame rate of, for example, 10 Hz to 30 Hz. An image generated from the data may be refreshed at a similar frame rate. Data may be acquired and displayed at different rates in other embodiments. For example, depending on the size of the volume and potential applications, ultrasonic data may be acquired at a frame rate of less than 10 Hz or greater than 30 Hz in some embodiments. For example, many applications involve acquiring ultrasonic data at a frame rate of 50 Hz. A memory 120 is included therein to store processing frames for acquiring data. In an exemplary embodiment, the memory 120 has sufficient capacity to store ultrasonic data frames acquired over a period of time that are at least a few seconds long. The data frames are stored in a manner that facilitates retrieval according to the order or time of acquisition thereof. The memory 120 may include any known data storage medium.

Optionally, the embodiments of the present invention may be carried out using a contrast agent. When an ultrasound contrast agent including microbubbles is used, enhanced images of anatomical structures and blood flow in the body are generated by contrast imaging. After acquiring data using the contrast agent, image analysis includes: separating a harmonic component from a linear component, enhancing the harmonic component, and generating an ultrasonic image by using the enhanced harmonic component. Separation of the harmonic component from the received signal is performed using an appropriate filter. The use of a contrast agent in ultrasonic imaging is well known to those skilled in the art, and therefore is not described in further detail.

In various embodiments of the present invention, data may be processed by the processor 116 through modules of other or different related modes (for example, B-mode, color Doppler, M-mode, color M-mode, spectral Doppler, elastography, TVI, strain, strain rate, and so on) to form 2D or 3D images or data. For example, one or a plurality of modules may generate B-mode, color Doppler, M-mode, color M-mode, spectral Doppler, elastography, TVI, strain, strain rate, a combination thereof, and so on. Image bundles and/or frames are stored, and timing information indicating the time when data is acquired in the memory may be recorded. The module may include, for example, a scan conversion module that performs scan conversion operations to convert image frames from a coordinate bundle space to display space coordinates. A video processor module may be provided that reads image frames from the memory and displays the image frames in real time while performing operation on a patient. The video processor module may store image frames in an image memory, read images from the image memory, and display the images. The ultrasonic imaging system 100 may be a console-based system, a laptop computer, a handheld or portable system, or any other configuration.

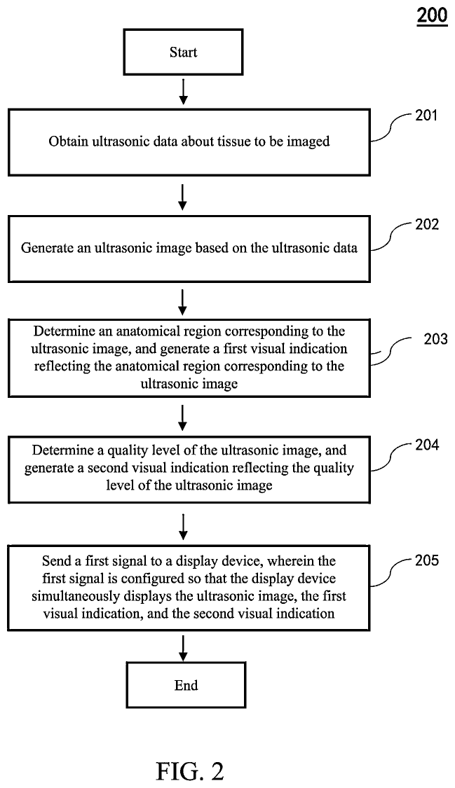

FIG. 2 is a flowchart of an ultrasonic imaging method 200 according to some embodiments of the present invention. Various modules in the flowchart represent steps that can be performed according to the method 200. Additional embodiments may perform the illustrated steps in a different order, and/or additional embodiments may include additional steps not shown in FIG. 2.

FIG. 2 is described in further detail below according to an exemplary embodiment. The method may be performed by the ultrasonic imaging system 100 shown in FIG. 1. For example, the method may be performed by the processor 116 in the ultrasonic imaging system 100.

In step 201, ultrasonic data about tissue to be imaged is obtained. The obtaining process may be implemented by the aforementioned processor 116. For example, the processor 116 may obtain from the probe 106 ultrasonic data acquired from a body part of a person to be scanned. Generally, ultrasonic signals may be sent by the probe 106 to the tissue to be imaged, and then ultrasonic echo signals from the tissue to be imaged are received by the probe 106. The processor 116 then can obtain ultrasonic data about the tissue to be imaged. The tissue to be imaged may be any human/animal tissue or organ. For example, the tissue to be imaged may be a liver, a kidney, a heart, a carotid artery, a breast, or the like, which will not be described herein again.

The aforementioned ultrasonic data may include 1D ultrasonic data, 2D ultrasonic data, 3D ultrasonic data, or 4D ultrasonic data. The ultrasonic data may be acquired and displayed in real time to serve as part of the real-time ultrasonic imaging process. Alternatively, in some other embodiments, the ultrasonic data may be acquired and processed in a first discrete time period, and then displayed after processing.

In step 202, an ultrasonic image is generated based on the ultrasonic data. The process may be accomplished by the processor 116. The image may be a 1D image, a 2D image, a 3D image, or a 4D image. The image may be generated from any mode of ultrasonic data. For example, the image may be a B-mode image, a color Doppler image, an M-mode image, a color M-mode image, a spectral Doppler image, an elastography image, a TVI image, or any other type of image generated from ultrasonic data. According to an embodiment, the image may be a still frame generated from ultrasonic data. According to other embodiments, the processor 116 may generate images from two or more different imaging modes based on the ultrasonic data. For example, in a VTI mode, the processor 116 may generate both a B-mode image and a spectral Doppler image based on the ultrasonic data. For example, in an IVC mode, the processor 116 may generate both a B-mode image and an M-mode image based on the ultrasonic data.

In step 203, an anatomical region corresponding to the ultrasonic image is determined, and a first visual indication reflecting the anatomical region corresponding to the ultrasonic image is generated. The process may also be implemented by the processor 116. The anatomical region is a specific position in the tissue to be imaged at which the ultrasonic image is acquired from the tissue to be imaged.

The method for determining the anatomical region corresponding to the ultrasonic image may be varied. In some embodiments, the anatomical region corresponding to the ultrasonic image may be directly determined by a neural network obtained by pre-training. For example, the ultrasonic image may be a 3D ultrasonic image. The processor 116 can directly determine, by the neural network, which anatomical region (for example, the left atrium) that the 3D ultrasonic image is obtained from. The neural network may be obtained by means of, for example, deep learning or machine learning, which will not be described herein again. Such an implementation can have a high degree of automation, and is applied to scans of different tissue to be imaged throughout the body.

In some other embodiments, the method for determining the anatomical region corresponding to the ultrasonic image may not reply on the ultrasonic image. For example, in an automatic or semi-automatic ultrasonic imaging system, the scanning trajectory or scanning angle of a probe is programmed and controlled by a processor. In such an example, the processor can know the position of the probe at any time, so as to directly obtain the anatomical region that the obtained ultrasonic image comes from. For example, in automatic breast ultrasound, the processor can directly know, according to the route of the probe, which region of the breast that the ultrasonic image comes from.

After the anatomical region corresponding to the ultrasonic image is determined, a first visual indication reflecting the anatomical region corresponding to the ultrasonic image may be generated. The first visual indication may be representation made in the form of text directly. However, in some scans of tissue to be imaged, it is difficult for textual representation to directly indicate the anatomical region corresponding to the ultrasonic image.

In some other embodiments, the first visual indication may include a visual indication of a position of the anatomical region corresponding to the ultrasonic image on the tissue to be imaged. For example, the entirety of the tissue to be imaged (for example, the heart, breast, liver, kidney, or carotid artery) may be represented using a graph, and an anatomical region corresponding to a generated ultrasonic graph is highlighted on the graph.

The graphic representation may exist in many manners. For example, a line may be used to outline a shape graph of the tissue to be imaged so as to facilitate direct intuitive determination of the user. The shape graph may be transparent, or may be of a certain color. Accordingly, the highlighting may also exist in various manners. For example, another color different from the color described above may be used to represent the anatomical region corresponding to the ultrasonic image. Alternatively, the anatomical region may also be highlighted by hatching or the like. In a word, the direct position of the anatomical region corresponding to the ultrasonic image on the tissue to be imaged is intuitively visually indicated, so that direct observation and determination of the user can be greatly facilitated.

In step 204, a quality level of the ultrasonic image is determined, and a second visual indication reflecting the quality level of the ultrasonic image is generated. The step may be implemented by the processor 116.

Specifically, the processor 116 may determine a target object acquisition quality level based on two or more different quality parameters. Alternatively, according to other implementation schemes, the processor 116 may determine an ultrasonic image acquisition quality level based on only a single quality parameter.

According to some implementation schemes, the quality parameters may include ultrasonic image quality parameters calculated from ultrasonic data, while in other implementations, the quality parameters may come from data including non-ultrasonic data. For example, the quality parameters may be acquired using a non-ultrasonic sensor. The quality parameters may include, for example, a noise level of the image, a measure of frame consistency over time, a signal strength, a view correctness measure, correctness of a flow pattern waveform, or any other parameter associated with object acquisition quality. Generally, a low noise level is related to high ultrasonic image acquisition quality, a small amount of probe motion is related to high ultrasonic image acquisition quality, a high measure of frame consistency over time is related to high ultrasonic image acquisition quality, and object size and shape (including roundness) is related to high ultrasonic image acquisition quality. The view correctness measure may be calculated by comparing an acquired image frame with a standard view using an image correlation technique. In some implementation, a neural network may be used to determine a matching degree of an acquired image frame with a standard view. The neural network may be obtained by training by means of deep learning, machine learning, or the like.

The ultrasonic image quality level may be determined by, for example, the noise level of the image. Specifically, threshold noise levels may be provided, and when the noise level does not exceed any threshold noise level, a first ultrasonic image quality level is determined, such as having an excellent quality level, while when the noise level is above a first threshold level but below a second threshold level, a second ultrasonic image acquisition quality level is determined, such as having an average quality level. Similarly, a noise level exceeding the second threshold level has a third ultrasonic image acquisition quality level, such as having a poor quality level. In some embodiments, three or more different quality levels may exist, for example, good, medium, poor. Alternatively, in some other embodiments, only two different quality levels may exist, for example, qualified or unqualified.

In yet another example, the ultrasonic image quality level is determined based on or in response to an amount of probe motion. In this example, the change in direction is continuously monitored by a sensor (such as an accelerometer) to determine the amount of probe movement. In this example, the quality level is inversely proportional to the amount of movement and changes over time.

In another example, the measure of frame consistency over time is used as an ultrasonic image quality parameter and a consistency range is determined by an algorithm. Based on the size of the range or the difference in frames over time. Based on the size of the range or variance between frames, a target object acquisition quality level is determined, wherein a small range indicates high quality, while a large range indicates low quality. Alternatively, an average variance from an average frame value is used, wherein increased variance indicates low quality, while decreased variance indicates high quality. Similarly, an average variance from a median frame value is used, wherein increased variance indicates low quality. Alternatively, in an implementation, a neural network is used to determine a target object quality level.

In another example, signal strength is used to determine the ultrasonic image quality level. In an example, a single threshold level is used. In this example, strength above a threshold strength level is considered as a high quality, while a signal at or below the threshold strength level is considered as a low quality.

In yet another example, a view correctness measure is calculated to determine the ultrasonic image quality level. In an example, an enhanced learning algorithm is used, wherein different weights are provided for different variables according to the accuracy of a checked reading. In an example, an interference level is one of the variables, the view correctness measure is another variable, and the signal strength is yet another variable. During iterative checks, a weight is applied for each variable. Specifically, when the reading is considered accurate during the check, the variable reading is assigned with a large weight when the reading is inaccurate. Thus, if the interference value is higher than a threshold, while the view correctness measure and the signal strength value are also lower than thresholds, and the reading is determined to be accurate, then the view correctness threshold and the signal strength threshold are assigned with high weights, while the interference threshold is assigned with a low weight. These new weights are then used to determine whether an accurate reading or determination is obtained in the next value iteration. Alternatively, the interference threshold may be increased in response to the accurate reading. Thus, the threshold may also vary through the iterative process.

In yet another example, correctness of a flow pattern waveform may be used. Likewise, an enhanced learning method may be used. Alternatively, different features, such as a slope, a peak-to-peak height, and the like, may be used and compared with previous measurement results to determine the ultrasonic image quality level.

In some examples, the determination of the quality parameters may further rely, at least in part, on direct determination of a quality level of an ultrasonic image generated by the ultrasonic imaging system. Direct determination of the quality level may be implemented by means of artificial intelligence. For example, it is determined by the neural network whether the ultrasonic image generated by the system has an artifact; it is determined by the neural network whether the ultrasonic image generated by the system is complete; it is determined by the neural network whether the scanning depth of the ultrasonic image generated by the system meets requirements, and so on. These quality parameters may be used in combination with the quality parameters in the above text for jointly determining the quality level of the ultrasonic image, so that the determination on the ultrasonic image quality level is more accurate and fits user needs.

Parameter indexes for determining the ultrasonic image quality level may be many and varied as listed above. The inventor has found that selecting the same quality parameter of ultrasonic image quality level for all tissue to be imaged may cause an inaccurate determination result. A user focuses on different things of ultrasonic images for different tissue to be imaged. For example, during a breast scan, scan completeness of mammary glands is one of the most important criteria for evaluating ultrasonic image quality. Carotid arteries do not have a glandular structure similar to that of mammary glands, and the scanning angle in a carotid artery scan will have a more important impact on image quality. Therefore, if the same criterion is used for the two different types of tissue to be imaged, the user's confidence in the accuracy of the indication provided by the present ultrasonic imaging method may be reduced.

In some embodiments of the present invention, automatic determination on the quality level of the ultrasonic image may be performed by using a corresponding neural network based on the tissue to be imaged. Different tissue to be imaged may have a different trained model. For example, for a breast ultrasound scan, the model may include some specific parameters. For example, completeness of mammary glands obtained from the ultrasonic image, whether a pressure value of the probe on the breast during acquisition of the ultrasonic image is suitable, whether the acquired image has an artifact, and conformance of the probe with respect to the breast during acquisition. These parameters may be assigned with different proportions to determine the overall ultrasonic image quality level. The aforementioned model may be specifically used for breast scans. However, when the scan object is the heart, carotid arteries, kidney, liver, or the like, automatic determination on quality levels of ultrasonic images of such tissues to be imaged may also be separately performed by using other corresponding neural networks. Before determination, the determination of the tissue to be imaged may be varied. For example, the tissue to be imaged may be selected by the user, or may be automatically determined by the ultrasonic imaging system 100, which will not be described herein again. In addition, in some embodiments, the quality level determination criteria may further be selected according to the anatomical region corresponding to the ultrasonic image.

After the quality level of the ultrasonic image is determined through the above example, a second visual indication reflecting the quality level of the ultrasonic image may be generated. The second visual indication may be arbitrary, with the function of enabling the user to intuitively understand whether the quality of the ultrasonic image obtained by the ultrasonic imaging system is qualified. The second visual indication is exemplary described below.

The second visual indication may be a color indication. The processor selects a color corresponding to the quality level based on the quality level of the ultrasonic image. The processor 116 may select from at least a first color and a second color, wherein the second color is different from the first color. According to an embodiment, the first color may represent a first ultrasonic image quality level, and the second color may represent second ultrasonic image quality. According to an embodiment, the first color may represent an ultrasonic image quality level in a first range, and the second color may represent an ultrasonic image quality level in a second range, wherein the second range does not overlap the first range. The first color may be, for example, green, and the ultrasonic image quality level in the first range may represent an acquisition quality level considered acceptable. The second color may be, for example, red, and the acquisition quality level in the second range may represent an ultrasonic image quality level considered unacceptable.

In addition, more than three colors may further be used to represent more than three different ultrasonic image quality levels. For example, a first color, for example, green, may represent a first quality level; a second color, for example, yellow, may represent a second quality level; and a third color, for example, red, may represent a third quality level. Alternatively, a first color may represent a quality level in a first range, a second color may represent a quality level in a second range, and a third color may represent a quality level in a third range. According to an embodiment, the quality level in the first range, the quality level in the second range, and the quality level in the third range may be discrete and non-overlapping ranges. According to other embodiments, more than three different colors may be used to represent various quality levels or various ranges of quality levels. Specifically, green may be a first color, which may be used to represent a high ultrasonic image quality level; red may be a second color, which may be used to represent a low ultrasonic image quality level; and yellow may be a third color, which may be used to represent a medium ultrasonic image quality level.

The correspondence between colors and ultrasonic image quality levels may not be intuitive. For example, a user having less experience or using the ultrasonic imaging system disclosed in the present invention for the first time is not necessarily able to intuitively understand which color represents a high ultrasonic image quality level and which color represents a low ultrasonic image quality level. In some embodiments, the second visual indication may reflect the ultrasonic image quality level in other manners.

The second visual indication may further be an icon indication. The processor selects an icon corresponding to the quality level based on the quality level of the ultrasonic image. The processor 116 may select from at least a first icon and a second icon, wherein the second icon is different from the first icon. Similar to the aforementioned color indication, the first icon may represent a first ultrasonic image quality level, and the second icon may represent second ultrasonic image quality. The first icon may represent an ultrasonic image quality level in a first range, and the second icon may represent an ultrasonic image quality level in a second range, wherein the second range does not overlap the first range.

The appearance of the first icon and the second icon may be configured to be easily visually distinguishable. In this way, the user can conveniently make intuitive determinations in the subsequent process to determine the quality level of the ultrasonic image. For example, the first icon may represent an acceptable ultrasonic image quality level, which may be “√”; and the second icon may represent a low ultrasonic quality level, which may be “×”. After viewing such conspicuous symbols, the user can make a direct determination on the ultrasonic image quality level.

In addition, more than three icon indications may further be used to represent more than three different ultrasonic image quality levels. For example, a first icon (for example, “√√”) may represent a first acquisition quality level; a second icon (for example, “√”) may represent a second acquisition quality level; and a third icon (for example, “×”) may represent a third acquisition quality level. Alternatively, the first icon may represent an acquisition quality level in a first range, a second icon may represent an acquisition quality level in a second range, and a third icon may represent an acquisition quality level in a third range. According to an embodiment, the image quality level in the first range, the image quality level in the second range, and the image quality level in the third range may be discrete and non-overlapping ranges. According to other embodiments, more than three different icons may further be used to represent various image quality levels or various ranges of image quality levels, which will not be described herein again.

In some other examples, the second visual indication may further be a combination of a color indication and an icon indication. In this way, a more noticeable indication can be given to the user in the subsequent process.

For example, the processor 116 may select a color and an icon corresponding to the quality level based on the quality level of the ultrasonic image. The processor 116 may select from at least a first icon having a first color and a second icon having a second color, wherein the second color is different from the first color, and the second icon is also different from the first icon. According to an embodiment, the first color may represent a first ultrasonic image quality level, and the second color may represent second ultrasonic image quality. According to an embodiment, the first color may represent an ultrasonic image quality level in a first range, and the second color may represent an ultrasonic image quality level in a second range, wherein the second range does not overlap the first range. The first icon having the first color may be, for example, green “√”, and the ultrasonic image quality level in the first range may represent an acquisition quality level considered acceptable. The second icon having the second color may be, for example, red “×”, and the acquisition quality level in the second range may represent an ultrasonic image quality level considered unacceptable. In addition, similar to the above description, more than three different icons having different colors may further be used to respectively represent more than three different ultrasonic image quality levels, which will not be described herein again.

On the basis of the aforementioned ultrasonic image, first visual indication, and second visual indication generated, a display may be controlled for display. Specifically, as shown in step 205, a first signal may be sent to a display device, wherein the first signal is configured so that the display device simultaneously displays the ultrasonic image, the first visual indication, and the second visual indication. The display device may be the display device 118 shown in FIG. 1. The simultaneous display means that the ultrasonic image, the first visual indication, and the second visual indication are simultaneously displayed on the same interface of the display device 118, thereby facilitating direct simultaneous observation of the ultrasonic image, the first visual indication, and the second visual indication by the user.

The aforementioned manner of simultaneous display may be exhibiting the ultrasonic image, the first visual indication, and the second visual indication on the display device 118 in any manner. The manner of exhibit display may include non-overlapping, partial overlapping, or overlapping display. In some embodiments, the second visual indication may be provided at an edge of the ultrasonic image. For example, an edge of an ultrasonic image with qualified quality may be set as a first color, and an edge of an ultrasonic image with unqualified quality may be set as a second color. Alternatively, an edge (for example, a corner) of an ultrasonic image with qualified quality may be set as a first icon, and an edge of an ultrasonic image with unqualified quality may be set as a second icon. Further, an edge (for example, a corner) of an ultrasonic image with qualified quality may be set as a first icon having a first color, and an edge of an ultrasonic image with unqualified quality may be set as a second icon having a second color. Such a configuration can ensure, on the one hand, that the user quickly corresponds an ultrasonic image to a quality level of the ultrasonic image, and on the other hand, that the second visual indication does not excessively interfere with the observation of the ultrasonic image.

In the present invention, a first visual indication is used to indicate an anatomical position corresponding to an ultrasonic image, a second visual indication is combined to indicate the quality of the ultrasonic image, and the first visual indication and the second visual indication are arranged together with the ultrasonic image. In the subsequent ultrasound scanning process, the user, on the one hand, can conveniently know whether a quality level of an ultrasonic image obtained by scanning meets requirements, and on the other hand, can intuitively know which position of tissue to be imaged that the ultrasonic image is taken from. In this way, when an ultrasonic scanning result at a certain position is unqualified, the user can perform a rescan at the position in a targeted manner.

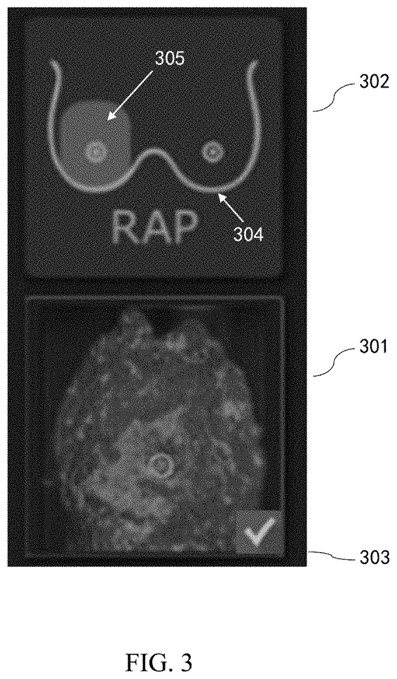

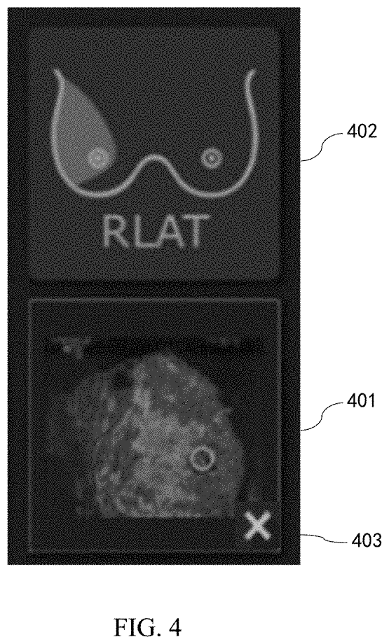

Some more specific exemplary description is provided below for the above embodiments, and reference may be made to FIG. 3 and FIG. 4 respectively. FIG. 3 shows a schematic diagram of an image in some embodiments of the present invention. FIG. 4 shows a schematic diagram of an image in some other embodiments of the present invention. The tissue to be imaged in FIGS. 3 and 4 is a human breast. First referring to FIG. 3, an ultrasonic image 301, a first visual indication 302, and a second visual indication 303 are simultaneously displayed in this example. The ultrasonic image 301 and the first visual indication 302 may be respectively displayed on the display device. The arrangement relationship of the ultrasonic image and the first visual indication is a vertical arrangement, and the first visual indication 302 is arranged above the ultrasonic image 301. It can be seen from FIG. 3 that the first visual indication 302 includes a profile graph 304 of breasts (the tissue to be imaged in this example), and an anatomical region view 305 corresponding to the ultrasonic image 301. The anatomical region view 305 is clearly marked in the profile graph 304, so as to facilitate intuitive observation by the user. The first visual indication 302 is schematically arranged above the ultrasonic image 301. In addition, a corner (specifically, the lower right corner) of the ultrasonic image 301 is overlaid with the second visual indication 303, for indicating the imaging quality of the ultrasonic image 301. In this example, the second visual indication 303 is an icon indication (specifically, “√”) having a color (specifically, green), which may be used to represent qualified ultrasonic image quality. On the one hand, the second visual indication 303 is provided on a corner of the ultrasonic image 301 without blocking the user's observation of the ultrasonic image 301. On the other hand, the second visual indication can also provide an intuitive and conspicuous identification to remind the user of the quality of the ultrasonic image 301.

Then referring to FIG. 4, another ultrasonic image 401, another first visual indication 402, and another second visual indication 403 are simultaneously displayed in this example. This example is generally similar to the example shown in FIG. 3. The difference is that the another second visual indication 403 in this example schematically describes another icon indication (specifically, “×”) having another color (specifically, red), which may be used to represent unqualified ultrasonic image quality. It can be seen that the visual indication enables the user to clearly determine the unqualified ultrasonic image quality, thereby facilitating making the next decision, for example, performing a rescan in combination with the anatomical structure indicated by the another first visual indication 402.

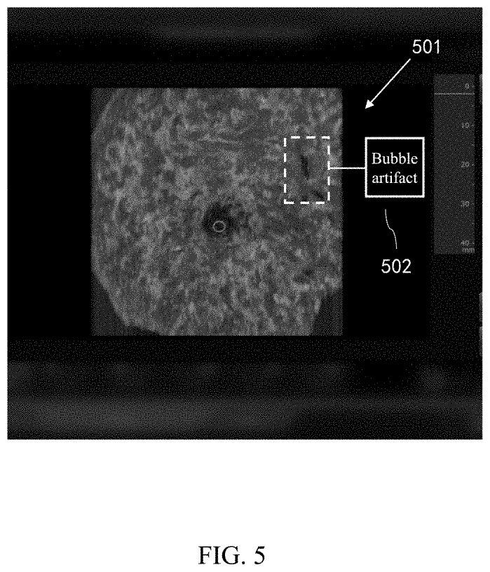

Displaying a plurality of images (for example, the aforementioned ultrasonic image, first visual indication, and second visual indication) on the same display device may make it difficult for the user to clearly view details of the ultrasonic image. Some embodiments of the present invention further provide a solution. Referring to FIG. 5, FIG. 5 shows a schematic diagram of an enlarged ultrasonic image 501 in some embodiments of the present invention. The enlarged ultrasonic image may be implemented by the following method: the processor sends a second signal to the display device in response to user input, wherein the second signal is configured so that the display device displays the enlarged ultrasonic image 501. The user input may be in any manner, for example, implemented by operating a keyboard, a trackball, a mouse, or a touch screen. In some non-limiting embodiments, the user input may also be implemented by means of speech input or the like, which will not be described herein again. For example, the user may send the user input to the processor by clicking on another ultrasonic image 401 shown in FIG. 4. The processor sends a second signal to the display device in response to the user input, so that the display device displays the enlarged ultrasonic image 501. The enlarged ultrasonic image 501 may be enlarged and displayed on top of the content displayed in the previous step, or may be displayed independently.

After enlargement, the user can observe details of the ultrasonic image more clearly. Especially in situations in which the displayed ultrasonic image has low quality, through enlargement and display, the user can know the cause of the low quality of the ultrasonic image more clearly, thereby facilitating improving the success rate of rescans.

In some other examples, the second signal is further configured so that the display device displays the enlarged ultrasonic image and a quality indication of the ultrasonic image. The quality indication may be the quality indication 502 shown in FIG. 5. The quality indication 502 can intuitively indicate the quality of the ultrasonic image according to the determination result of the ultrasonic image quality level in the aforementioned step. For example, the quality indication may indicate the region of an image quality defect (the position indicated by the dashed box in FIG. 5). Further, the cause of the image quality defect may be indicated by text (“bubble artifact” indicated by the solid box in FIG. 5). Alternatively, the quality indication may be a combination of the two, showing both the position of the region of the image quality defect and the cause of the image quality defect. The quality indication 502 can more intuitively inform the user how to improve ultrasound scans.

In addition, the processor may further be configured to receive another user input so that the enlarged ultrasonic image returns to the display state in the previous step, which will not be described herein again.

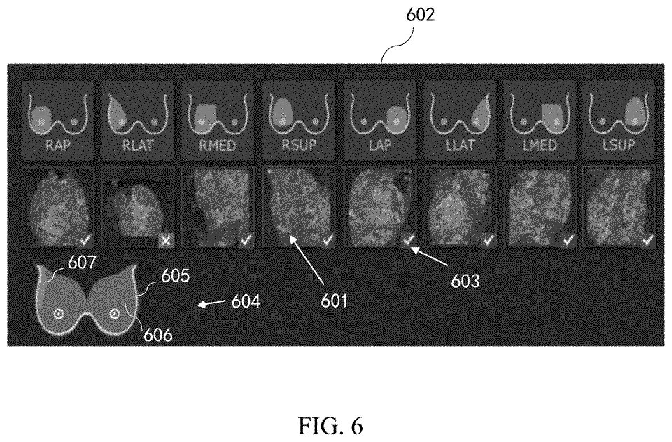

In some application scenarios, the user may need to determine whether the scan of the tissue to be imaged is completed. For example, whether acquisition of each anatomical plane of the tissue to be imaged is complete and meets requirements. Some embodiments of the present invention illustrate an indication for scan completeness of the tissue to be imaged. Referring to FIG. 6, an image including an indication for scan completeness of the tissue to be imaged in some embodiments of the present invention is illustrated. In some embodiments, the image may include a plurality of ultrasonic images 601, a plurality of first visual indications 602 respectively reflecting an anatomical region corresponding to each of the plurality of ultrasonic images 601, and a plurality of second visual indications 603 respectively reflecting a quality level of each of the plurality of ultrasonic images 601.

The manner of generating and displaying the aforementioned plurality of ultrasonic images 601, the plurality of first visual indications 602, and the plurality of second visual indications 603 can be in reference to any embodiment described above and will not be described herein again. Different from the aforementioned embodiment, in this embodiment, the plurality of ultrasonic images 601 are respectively acquired from the same tissue to be imaged. Specifically, the plurality of ultrasonic images 601 are respectively acquired from different positions of the same tissue to be imaged, for example, acquired from different positions of a breast. Such arrangement can reflect the quality of all ultrasonic images acquired from the entire tissue to be imaged and acquisition positions more intuitively, thereby providing more intuitive display to the user.

Further, in some examples, a third visual indication is further included. The third visual indication may be obtained by the processor according to the following steps: generating, according to the quality level of each of the plurality of ultrasonic images and the anatomical region corresponding to each of the plurality of ultrasonic images, a third visual indication reflecting scan completeness of the tissue to be imaged where the anatomical regions arelocated, wherein the first signal is further configured so that the display device simultaneously displays the ultrasonic images, the first visual indications, the second visual indications, and the third visual indication. Specifically, referring to FIG. 6, the third visual indication 604 may be generated by the processor according to the quality level of each of the plurality of ultrasonic images 601 and the anatomical region corresponding to each of the plurality of ultrasonic images, and used to reflect scan completeness of the tissue to be imaged where the anatomical region is located. For example, when a quality level of one of the plurality of ultrasonic images 601 is determined to be unqualified, it can be determined that an anatomical region corresponding to the ultrasonic image is not scanned. When a quality level of another ultrasonic image is determined to be qualified, it can be determined that an anatomical region corresponding to the ultrasonic image is already scanned. When a required anatomical region does not correspond to an ultrasonic image, it can be determined that the region is not scanned. According to the above method, the scan completeness of the tissue to be imaged can be determined, and further shown by the third visual indication 604. The manner of showing the third visual indication 604 may be varied. For example, a complete tissue profile 605 of the tissue to be scanned may be represented, and a region 606 for which the scan is completed is shown by one color, and a region 607 for which the scan is not completed is shown by another color. In this way, the user can intuitively determine which region has not been completely scanned, and then determine whether to perform a rescan and which part needs to be rescanned. This greatly improves the scanning efficiency of the user and the targeted second scan. The third visual indication 604 in FIG. 6 is applied to the plurality of ultrasonic images 601, and is also applicable to the single ultrasonic image shown in FIGS. 3 and 4.

Some embodiments of the present invention further provide an ultrasonic imaging system. The system may be shown in FIG. 1, or may be any other system. The system includes: a probe, configured to acquire ultrasonic data; and a processor, configured to perform the method in any of the embodiments described above. The system further includes a display device configured to receive a signal from the processor for display.

Some embodiments of the present invention further provide a non-transitory computer-readable medium storing a computer program, wherein the computer program has at least one code segment, and the at least one code segment is executable by a machine so that the machine performs steps of the method in any of the embodiments described above.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. An ultrasonic imaging method, comprising:
obtaining ultrasonic data about tissue to be imaged;
generating an ultrasonic image based on the ultrasonic data;
determining an anatomical region corresponding to the ultrasonic image, and generating a first visual indication reflecting the anatomical region corresponding to the ultrasonic image;
determining a quality level of the ultrasonic image, and generating a second visual indication reflecting the quality level of the ultrasonic image; and
sending a first signal to a display device, wherein the first signal is configured so that the display device simultaneously displays the ultrasonic image, the first visual indication, and the second visual indication.

2. The ultrasonic imaging method according to claim 1, wherein
said determining the quality level of the ultrasonic image comprises: performing automatic determination on the quality level of the ultrasonic image by using a corresponding neural network based on the tissue to be imaged.

3. The ultrasonic imaging method according to claim 1, wherein
the second visual indication comprises at least one of a color indication and an icon indication.

4. The ultrasonic imaging method according to claim 1, wherein
the second visual indication is provided at an edge of the ultrasonic image.

5. The ultrasonic imaging method according to claim 1, wherein
the first visual indication comprises a visual indication of a position of the anatomical region corresponding to the ultrasonic image on the tissue to be imaged.

6. The ultrasonic imaging method according to claim 5, further comprising:
generating, according to the quality level of the ultrasonic image and the anatomical region corresponding to the ultrasonic image, a third visual indication reflecting scan completeness of the tissue to be imaged where the anatomical region is located, wherein
the first signal is further configured so that the display device simultaneously displays the ultrasonic image, the first visual indication, the second visual indication, and the third visual indication.

7. The ultrasonic imaging method according to claim 1, further comprising:
sending a second signal to the display device in response to user input, wherein the second signal is configured so that the display device displays an enlarged ultrasonic image.

8. The ultrasonic imaging method according to claim 7, wherein
the second signal is further configured so that the display device displays the enlarged ultrasonic image and a quality indication of the ultrasonic image.

9. The ultrasonic imaging method according to claim 1, wherein
the ultrasonic image comprises a plurality of ultrasonic images;
the first visual indication comprises a plurality of first visual indications separately reflecting an anatomical region corresponding to each of the plurality of ultrasonic images; and
the second visual indication comprises a plurality of second visual indications separately reflecting a quality level of each of the plurality of ultrasonic images.

10. The ultrasonic imaging method according to claim 9, wherein
the anatomical regions corresponding to the plurality of ultrasonic images come from the same tissue to be imaged.

11. The ultrasonic imaging method according to claim 9, further comprising:
generating, according to the quality level of each of the plurality of ultrasonic images and the anatomical region corresponding to each of the plurality of ultrasonic images, a third visual indication reflecting scan completeness of the tissue to be imaged where the anatomical regions are located, wherein
the first signal is further configured so that the display device simultaneously displays the ultrasonic images, the first visual indications, the second visual indications, and the third visual indication.

12. An ultrasonic imaging system, comprising:
a probe, configured to acquire ultrasonic data;
a display device, configured to receive a signal from the processor for display; and
a processor, wherein the processor is configured to:
obtain ultrasonic data about tissue to be imaged;
generate an ultrasonic image based on the ultrasonic data;
determine an anatomical region corresponding to the ultrasonic image, and generate a first visual indication reflecting the anatomical region corresponding to the ultrasonic image;
determine a quality level of the ultrasonic image, and generate a second visual indication reflecting the quality level of the ultrasonic image; and
send a first signal to the display device, wherein the first signal is configured to cause the display device to simultaneously display the ultrasonic image, the first visual indication, and the second visual indication.

13. The ultrasonic imaging system of claim 12, wherein the processor is configured to dertermind the quality level of the ultrasonic image automatically by using a corresponding neural network based on the tissue to be imaged.

14. The ultrasonic imaging system of claim 12, wherein the second visual indication comprises at leat one of a color indication and an icon indication.

15. The ultrasonic imaging system of claim 12, wherein the second visual indication is provided at an edge of the ultrasonic image.

16. The ultrasonic imaging system of claim 12, wherein the first visual indication comprises a visual indication of a position of the anatomical region corresponding to the ultrasonic image on the tissue to be imaged.

17. The ultrasonic imaging system of claim 12, wherein the processor is further configured to:
generate, accordimg to the quality level of the ultrasonic image and the anatomical region corresponding to the ultrasonic image, a third visual indication reflection scan completeness of the tissue to be imaged where the anatomica region is located, wherein
the first signal signal is further configured to cause the display device to simultaneously display the ultrasonic image, the first visual indication, the second visual indication, and the third visual indication.

18. The ultrasonic imaging system of claim 12, wherein the processor is further configured to send a second signal to the display device in response to user input, wherein the second signal is configured to cause the display device to display an enlarged ultrasonic image.

19. The ultrasonic imaging system of claim 18, wherein the second signal is further configured to cause the display device to display the enlarged ultrasonic image and a quality indication of the ultrasonic image.

20. The ultrasonic imaging system of claim 12, wherein:
the ultrasonic image comprises a plurality of ultrasonic images;
the first visual indication comprises a plurality of first visual indications separately reflecting an anatomical region corresponding to each of the plurality of ultrasonic images; and
the second visual indication comprises a plurality of second visual indications separately reflecting a quality level of each of the plurality of ultrasonic images.
